(19) United States
(12) Patent Application Publication
(10) Pub. No.: US 2018/0315495 A1
(43) Pub. Date: Nov. 1, 2018

(54) Ensuring treatment initiation

(71) Applicant: Spring Care, Inc., Brooklyn, NY (US)

(72) Inventors: Adam Chekroud, New Haven, CT (US); Abhishek Chandra, Brooklyn, NY (US)

(21) Appl. No.: 15/965,918

(22) Filed: Apr. 28, 2018

Related U.S. Application Data
(60) Provisional application No. 62/491,660, filed on Apr. 28, 2017.

(51) Int. Cl. G16H 20/10; G16H 50/30; G16H 80/00

(52) U.S. Cl. CPC G16H 20/10; G16H 80/00; G16H 50/30

(57) ABSTRACT

Methods and systems for ensuring patient initiation of a treatment regime. A patient may provide one or more patient parameters including, for example, medical history of the patient, socio-demographic data of the patient, and behavioral health symptoms of the patient. An engagement model may then predict whether the patient is likely to initiate a treatment regime, as well as the reasons why the patient may not initiate the treatment regime. Clinicians may then take appropriate steps to ensure the patient initiates treatment.

### CROSS-REFERENCE TO RELATED APPLICATIONS

This application claims priority to and the benefit of U.S. Provisional Patent Application No. 62/491,660, filed on Apr. 28, 2017, which is hereby incorporated by reference in its entirety.

### TECHNICAL FIELD

Embodiments described herein generally relate to systems and methods for treating a patient and, more particularly but not exclusively, to systems and methods for ensuring patient initiation of a treatment regime.

### BACKGROUND

Despite the availability of safe and effective treatments for mental illnesses and other conditions, many people who are diagnosed with a condition do not return for treatment after the diagnosis is made. This may ultimately lead the patient to go untreated.

A need exists, therefore, for systems and methods for ensuring patient initiation of a treatment.

### SUMMARY

This summary is provided to introduce a selection of concepts in a simplified form that are further described below in the Detailed Description section. This summary is not intended to identify or exclude key features or essential features of the claimed subject matter, nor is it intended to be used as an aid in determining the scope of the claimed subject matter.

In one aspect, embodiments relate to a system for ensuring patient initiation of a treatment regime. The system includes a user interface for receiving at least one patient parameter; and a processor executing instructions stored on a memory and providing an engagement model, wherein the engagement model is configured to at least receive the at least one patient parameter, predict whether a patient will initiate a treatment regime based on the at least one patient parameter, and elevate the patient for further examination upon predicting the patient will not initiate the treatment regime.

In some embodiments, the user interface executes on a user device, the processor executes on a server remote from the user device, and the at least one patient parameter is cached on the user device until a connection is established between the user device and the server.

In some embodiments, the user interface is further configured to present a questionnaire to the patient to receive the at least one patient parameter from the patient.

In some embodiments, the engagement model is further configured to generate a report of the prediction, the generated report including at least one reason why the patient will not initiate the treatment regime. In some embodiments, the generated report further includes at least one recommended treatment regime based on the at least one reason why the patient will not initiate the treatment regime.

In some embodiments, the at least one patient parameter includes at least one of medical history of the patient, socio-demographic data of the patient, and behavioral health symptoms of the patient.

In some embodiments, the user interface is further configured to receive data regarding the patient's compliance with the treatment regime.

In some embodiments, the prediction of whether the patient will initiate the treatment regime is further based on data from an electronic medical record associated with the patient.

In some embodiments the engagement model predicts whether the patient will initiate a treatment regime by: assigning a value to each of the plurality of patient parameters, summing the assigned values to calculate a score, and predicting whether the patient will initiate the treatment regime based on the score.

According to another aspect, embodiments relate to a method for ensuring patient initiation of a treatment regime. The method includes receiving at least one patient parameter using a user interface, providing the at least one patient parameter to a processor executing instructions stored on a memory and providing an engagement model, and receiving a prediction regarding whether the patient will initiate a treatment regime based on the at least one patient parameter.

In some embodiments, the user interface executes on a user device, the processor executes on a server remote from the user device, and the at least one patient parameter is cached on the user device until a connection is established between the user device and the server.

In some embodiments, the method further includes presenting, using the user interface, a questionnaire to the patient to receive the at least one patient parameter.

In some embodiments, the received prediction indicates the patient will not initiate the treatment regime, and the method further comprises receiving a report including at least one reason why the patient will not initiate the treatment regime. In some embodiments, the received report further includes at least one recommended treatment regime based on the at least one reason why the patient will not initiate the treatment regime.

In some embodiments, the at least one patient parameter includes at least one of medical history of the patient, socio-demographic data of the patient, and behavioral health symptoms of the patient.

In some embodiments, the method further includes receiving data regarding the patient's compliance with the treatment regime using the user interface.

In some embodiments, the prediction of whether the patient will initiate the treatment regime is further based on data from an electronic medical record associated with the patient.

In some embodiments, the engagement model generates the prediction regarding whether the patient will initiate the treatment regime by assigning a value to each of the plurality of patient parameters, summing the assigned values to calculate a score, and predicting whether the patient will initiate the treatment regime based on the score.

According to another aspect, embodiments relate to a computer readable medium containing computer readable instructions for performing a method for ensuring patient initiation of a treatment. The medium includes computer-readable instructions for receiving at least one patient parameter using a user interface, computer-readable instructions for providing the at least one patient parameter to a processor executing instructions stored on a memory and providing an engagement model, and computer-readable instructions for receiving a prediction regarding whether the patient will initiate a treatment regime based on the at least one patient parameter.

In some embodiments the at least one patient parameter includes at least one of medical history of the patient, socio-demographic data of the patient, and behavioral health symptoms of the patient.

### BRIEF DESCRIPTION OF DRAWINGS

Non-limiting and non-exhaustive embodiments of the invention are described with reference to the following figures, wherein like reference numerals refer to like parts throughout the various views unless otherwise specified.

FIG. 1 illustrates a workflow of a method for ensuring patient initiation of a treatment regime in accordance with one embodiment;

FIG. 2 illustrates a system for ensuring patient initiation of a treatment regime in accordance with one embodiment;

FIG. 3 illustrates a workflow of the components of the system of FIG. 2 in accordance with one embodiment;

FIG. 4 presents a table of certain variables used for predicting whether a patient will initiate a treatment regime in accordance with one embodiment;

FIGS. 5a-c illustrate variables associated with reasons for not initiating a treatment regime in accordance with one embodiment;

FIG. 6 depicts a plot relating to a patient that is predicted to not initiate a treatment regime in accordance with one embodiment;

FIG. 7 depicts a plot relating to a patient that is predicted to initiate a treatment regime in accordance with one embodiment; and

FIG. 8 depicts a flowchart of a method for ensuring patient initiation of a treatment regime in accordance with one embodiment.

### DETAILED DESCRIPTION

Various embodiments are described more fully below with reference to the accompanying drawings, which form a part hereof, and which show specific exemplary embodiments. However, the concepts of the present disclosure may be implemented in many different forms and should not be construed as limited to the embodiments set forth herein; rather, these embodiments are provided as part of a thorough and complete disclosure, to fully convey the scope of the concepts, techniques and implementations of the present disclosure to those skilled in the art. Embodiments may be practiced as methods, systems or devices. Accordingly, embodiments may take the form of a hardware implementation, an entirely software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one example implementation or technique in accordance with the present disclosure. The appearances of the phrase “in one embodiment” in various places in the specification are not necessarily all referring to the same embodiment. The appearances of the phrase “in some embodiments” in various places in the specification are not necessarily all referring to the same embodiments.

Some portions of the description that follow are presented in terms of symbolic representations of operations on non-transient signals stored within a computer memory. These descriptions and representations are used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices. Portions of the present disclosure include processes and instructions that may be embodied in software, firmware or hardware, and when embodied in software, may be downloaded to reside on and be operated from different platforms used by a variety of operating systems.

The present disclosure also relates to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each may be coupled to a computer system bus. Furthermore, the computers referred to in the specification may include a single processor or may be architectures employing multiple processor designs for increased computing capability.

The processes and displays presented herein are not inherently related to any particular computer or other apparatus. Various general-purpose systems may also be used with programs in accordance with the teachings herein, or it may prove convenient to construct more specialized apparatus to perform one or more method steps. The structure for a variety of these systems is discussed in the description below. In addition, any particular programming language that is sufficient for achieving the techniques and implementations of the present disclosure may be used. A variety of programming languages may be used to implement the present disclosure as discussed herein.

In addition, the language used in the specification has been principally selected for readability and instructional purposes and may not have been selected to delineate or circumscribe the disclosed subject matter. Accordingly, the present disclosure is intended to be illustrative, and not limiting, of the scope of the concepts discussed herein.

Various embodiments of the systems and methods described herein provide a way for clinicians to better understand whether a patient is likely to initiate a prescribed and/or recommended treatment regime. Particularly, the systems and methods described herein may predict whether a patient will initiate a treatment for one or more behavioral health conditions, such as a mental disorder.

In addition to predicting whether a patient will initiate a treatment regime, the systems and methods described herein may also provide reasons for why a particular patient may not initiate the treatment regime. Accordingly, the predictive features described can be leveraged to focus health system resources on patients that are at risk of not initiating their treatment regime(s), and ultimately used to inform efforts toward universal treatment.

In the context of the present application, the term “treatment regime” may refer to any clinician-recommended and/or clinician-prescribed steps to treat a patient's condition(s). A treatment regime may include or otherwise involve taking an antidepressant or pursuing psychiatric evaluation, for example. Treatment regimes may also include participating in exercise regimes, spiritual services, religious services, body wellness programs, peer counseling programs, or the like. The above list of treatment regimes are merely exemplary and other types of treatment regimes may be considered.

In addition to ensuring patient initiation of a treatment regime, the systems and methods described herein may also/alternatively be used to ensure compliance with a treatment regime. Compliance, medically speaking, may begin once a patient has initiated a treatment regime.

To predict patient initiation of a treatment regime, the systems and methods described herein may consider various types of patient parameters. These parameters may include, but are not limited to, a patient's socio-demographics, a patient's behavioral health symptoms, and a patient's lifetime medical history. Additionally, these patient parameters may be analyzed in conjunction with data from a patient's medical record.

Socio-demographic information considered may include the patient's, age, sex, race, number of children, education, county type (e.g., large metro, small metro, non-metro), family household income, and whether the patient is covered by Medicare, Medicaid/CHIP, or by private health insurance. The above list is merely exemplary and other types of socio-demographic information may be considered.

Behavioral health information may include data related to various mental health diagnostic scales, depression or other symptom scales, and/or distress scales. Behavioral health information may also include self-reported endorsements of the patient having thought about, planned, or attempted suicide. This type of behavioral health information is merely exemplary and other types of behavioral health information may be considered.

Medical history information may include factors such as in how many of the last 30 days a patient smoked cigarettes, the number of times the patient as in the emergency room within the past year, the patient's self-reported overall health states (e.g., 1=“excellent” to 5=“poor”). The medical history information may also include whether the patient has ever been diagnosed with anxiety disorder, asthma, bronchitis, liver cirrhosis, diabetes, heart disease, hepatitis, high blood pressure, pneumonia, STD, sinusitis, sleep apnea, stroke, or ulcers. The above list is merely exemplary and other types of medical history information may be considered.

To make the process more convenient for patients and clinicians, the systems and methods described herein may present a questionnaire to the patient. The questionnaire may therefore inform the patient what types of data are required and therefore provide the patient with an easy-to-use means for providing the relevant patient parameters.

An ultimate goal for the systems and methods described herein may be to anticipate which individuals will not initiate treatment (and therefore provide for more concerted outreach), and then estimate how likely the patients are likely to accept various treatment options. The levels of risk can be relayed to appropriate clinicians and/or case managers to help foster shared decision making while minimizing barriers to initiating treatment. In other words, clinicians or other authorized personnel (“clinicians”) may leverage this information to take steps to ensure higher patient initiation of treatment regimes. Clinicians may discuss results with the patients, offer potential workarounds or alternatives to the treatment, and/or use the results to profile their patient population and deliver interventions to improve initiation (and compliance) with the treatment regimes.

For example, patients predicted to not initiate a treatment regime may be elevated for further examination. Clinicians may then take steps to specifically reduce any treatment-related stigmas, provide further education to the patient, and provide other types of support with respect to social determinants of health.

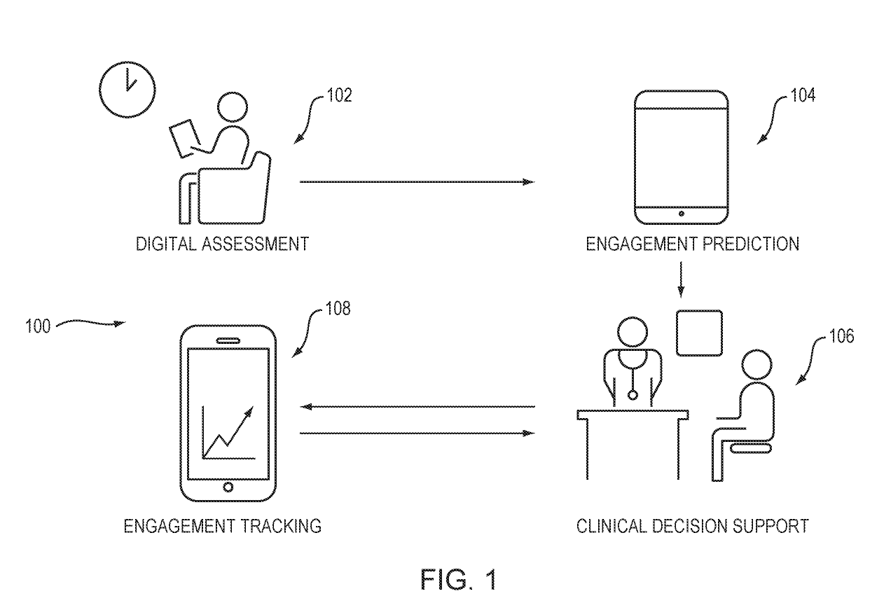

FIG. 1 illustrates a workflow 100 of the systems and methods described herein in accordance with some embodiments. Step 102 involves the digital assessment of a patient's health. This may include the patient's mental health. Step 102 may be performed by having a patient complete an administered questionnaire with questions regarding the patient's health.

Step 104 involves treatment initiation prediction. Based on the received patient parameters, an engagement model described herein may predict whether the patient is likely to initiate a treatment regime.

Step 106 involves clinical decision support. Clinicians may review the patient data as well as the prediction from the engagement model. For example, clinicians may review a prediction that a certain patient is unlikely to initiate a treatment regime, as well as one or more reasons the patient is unlikely to initiate the treatment regime. Clinicians may then take steps to make it at least more likely the patient will initiate the treatment regime.

Step 108 involves engagement/compliance tracking. The systems and methods described herein may continuously receive data regarding how well the patient is complying with the treatment regime (including whether or not the patient has in fact initiated the treatment regime). This information may help determine the accuracy of the predictions and further refine statistical algorithm(s) underlying the predictive capabilities of the system.

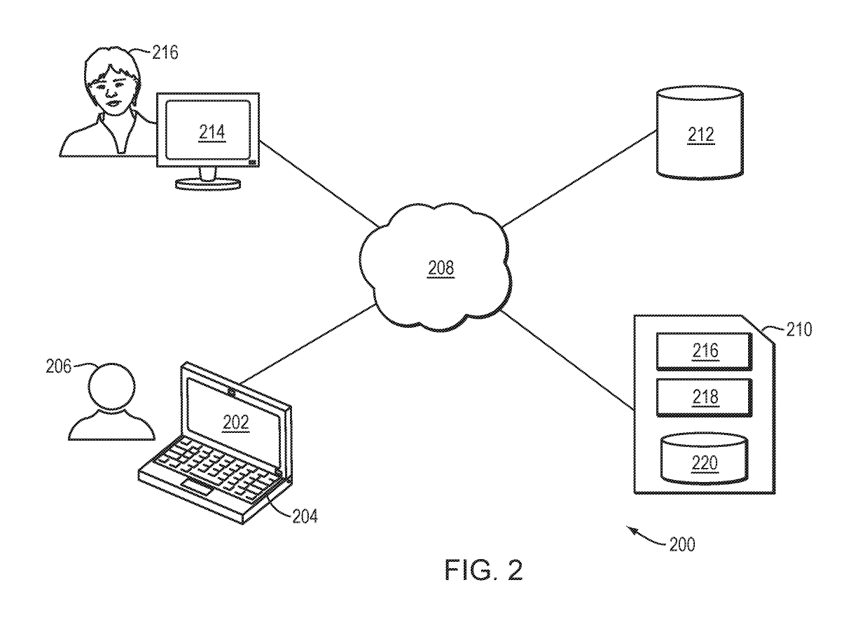

FIG. 2 illustrates a system 200 for ensuring patient initiation of a treatment regime in accordance with one embodiment. The system 200 may include a user interface 202 executing on a user device 204 to at least enable a patient 206 to enter at least one patient parameter.

The user device 204 may be connected to or otherwise in communication with a network 208 and be in further communication with a server 210, one or more databases 212 storing the patient's electronic medical record (EHR), and a clinician device 214 for use by a clinician 216.

The user interface 202 may be configured to enable the patient 206 to input one or more patient parameters. In some embodiments, the user interface 202 may present a questionnaire to prompt the patient 206 to input specific types of data.

The user interface 202 may be configured as a responsive web application frontend or a native mobile application. The application may communicate with the server 210 over SSL to ensure encryption of all data in transit.

Additionally, patient data and responses to any questionnaires may be cached on the user device 204 (e.g., either in secure storage on iOS, or signed cookies in a web browser of the patient's personal user device). This ensures that patient data is transmitted securely, and that the data is cached on the user device 204 until a connection is established between the user device 204 and the server 210. Additionally, the data may be cached on the user device 204 until the connection between the user device 204 and the server 210 is stable.

To further promote security, the cache may be invalidated every time a new patient starts using the user device 204 (e.g., if the user device 204 is located in the clinician's office). Or, in the case of a personal device, the cache may be invalidated when the patient logs out/off of their own device. This ensures that only one patient's information is cached at a time.

The user device 204 may be any suitable input/output device that allows a patient 206 to input various types of patient-related data. The user device 204 may be configured as a PC, a laptop, a tablet, a smartwatch, a smartphone, a kiosk, or the like. The exact configuration or implementation of the user device 204 may vary as long as the features of various embodiments of the systems and methods described herein can be accomplished.

In addition to allowing the patient 206 to enter at least one patient parameter, e.g., before diagnosis or before treatment is prescribed, the user interface 202 may allow the patient 206 to continuously input data regarding their compliance/progress with respect to a treatment regime. Accordingly, the clinician 216 can receive this information to verify that the patient 206 has indeed initiated a treatment regime and is also complying with the treatment regime.

For example, a patient 206 can be regularly prompted to complete follow-on questionnaires that ask them about their compliance with their prescribed treatment regime. These responses can be forwarded to clinicians 216 as a data point for further action (such as a follow-up appointment) and learning.

Moreover, a patient's responses can be used to further augment the engagement model to iteratively improve the prediction accuracy for the patient and future patients. This may help develop subsequent machine learning algorithms that incorporate temporal dynamics of the treatment engagement process.

The user device 204 may also be positioned at various locations for the patient's convenience. For example, the user device 204 may be a device located within the patient's home so that the patient 206 can enter patient parameters at their convenience (e.g., before traveling to a clinician's office). Similarly, as the user device 204 may be implemented as a smartphone, the user device 204 may be carried by patient 206 and used at the patient's convenience (e.g., as the patient 206 is traveling to the clinician's office).

Additionally or alternatively, the user device 204 may be located at the clinician's office (e.g., as a kiosk, PC, tablet, or the like). In these embodiments, the patient 206 may provide the relevant patient parameters while at the clinician's office (e.g., in the waiting room or in the examination room of the clinician's office).

The network(s) 208 may link the various components with various types of network connections. The network(s) 208 may be comprised of, or may interface to, any one or more of the Internet, an intranet, a Personal Area Network (PAN), a Local Area Network (LAN), a Wide Area Network (WAN), a Metropolitan Area Network (MAN), a storage area network (SAN), a frame relay connection, an Advanced Intelligent Network (AIN) connection, a synchronous optical network (SONET) connection, a digital T1, T3, E1, or E3 line, a Digital Data Service (DDS) connection, a Digital Subscriber Line (DSL) connection, an Ethernet connection, an Integrated Services Digital Network (ISDN) line, a dial-up port such as a V.90, a V.34, or a V.34bis analog modem connection, a cable modem, an Asynchronous Transfer Mode (ATM) connection, a Fiber Distributed Data Interface (FDDI) connection, a Copper Distributed Data Interface (CDDI) connection, or an optical/DWDM network.

The network or networks 208 may also comprise, include, or interface to any one or more of a Wireless Application Protocol (WAP) link, a Wi-Fi link, a microwave link, a General Packet Radio Service (GPRS) link, a Global System for Mobile Communication G(SM) link, a Code Division Multiple Access (CDMA) link, or a Time Division Multiple access (TDMA) link such as a cellular phone channel, a Global Positioning System (GPS) link, a cellular digital packet data (CDPD) link, a Research in Motion, Limited (RIM) duplex paging type device, a Bluetooth radio link, or an IEEE 802.11-based link.

The server 210 may include a processor 216 executing instructions stored on memory 218 to provide an engagement model. The server 210 may also include an application database 220 for at least storing outputs from the engagement model.

The processor 216 may be any hardware device capable of executing instructions stored on the memory 218 to provide the engagement model. The processor 216 may include a microprocessor, a field programmable gate array (FPGA), an application-specific integrated circuit (ASIC), or other similar devices. In some embodiments, such as those relying on one or more ASICs, the functionality described as being provided in part via software may instead be configured into the design of the ASICs and, as such, the associated software may be omitted. The processor 216 may be configured as part of the user device 204 (e.g., a laptop), located at some remote location (such as on the server 210), or part of the clinician device 214.

The memory 218 may be L1, L2, L3 cache or RAM memory configurations. The memory 218 may include non-volatile memory such as flash memory, EPROM, EEPROM, ROM, and PROM, or volatile memory such as static or dynamic RAM, as discussed above. The exact configuration/type of memory 218 may of course vary as long as instructions for ensuring patient compliance with a treatment regime can be executed to accomplish the various features described herein.

The one or more databases 212 may store various types of patient-related data, such as the patient's EHR. This data may be analyzed in conjunction with the received patient parameter(s) to accomplish the features of various embodiments described herein.

Data regarding the patient (such as the output of the engagement model as to whether the patient is predicted to initiate the treatment regime) may be communicated to the clinician device 214. The clinician 216 may review this data before or after meeting with the patient 206 to determine the most favorable treatment options for the patient 206 and therefore aid in clinical decision support.

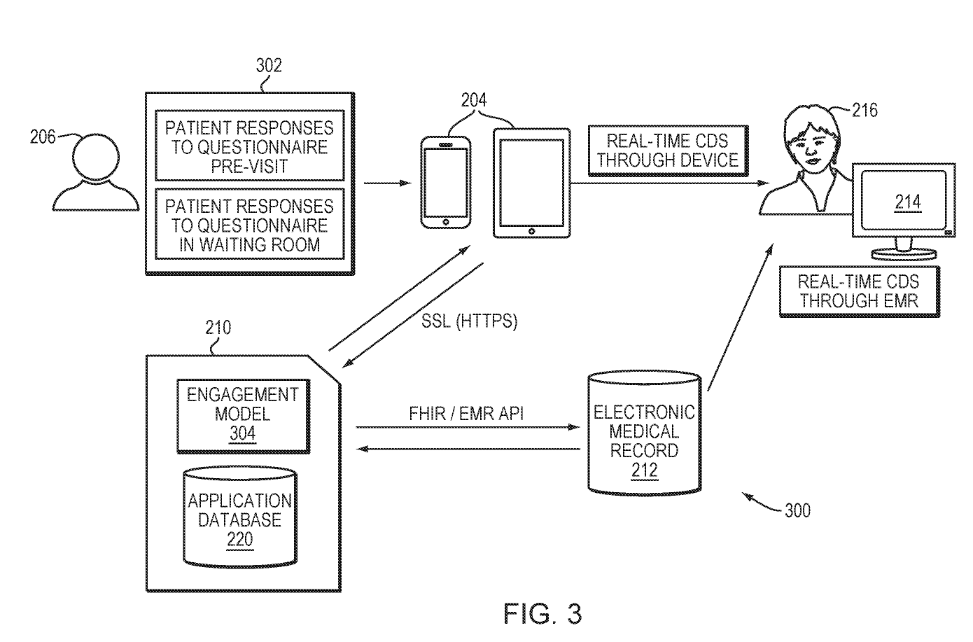

FIG. 3 presents an exemplary workflow 300 of the components of the system 200 of FIG. 2 in accordance with one embodiment. The patient 206 may provide responses 302 to, for example, a questionnaire either before visiting a clinician's office and/or while at a clinician's office. These responses may be supplied to the user device 204.

The responses may be directly communicated to the clinician's device 214 (or a device otherwise accessible by an appropriate clinician) for viewing by the clinician 216. This allows for clinical decision support monitoring in at least substantially real time.

The responses may additionally or alternatively be communicated to the server 210. The data may be communicated over SSL to ensure encryption of all data in transit.

Specifically, the data may be communicated to an engagement model 304. The engagement model 304 may be provided by a processor executing instructions stored on a memory such as the processor 216 and memory 218 of FIG. 2.

The engagement model 304 may be previously trained on data from a variety of sources. For example, the model may be trained on aggregated data from the U.S. National Survey on Drug Use and Health series between 2008 and 2014, and data collected from adults diagnosed with depression by a healthcare provider in the 12 months prior to the survey. This data source is merely exemplary, and the engagement model 304 may be trained on other data sources, whether available now or compiled hereafter.

The engagement model 304 may be hosted on a cloud-based web application with a front-end and an Application Programming Interface (API) to receive patient input from, e.g., a web browser executing on a user device. The application may store patient responses in the application database 220 and/or communicate this data to the patient's EHR in one or more databases 212. Accordingly, clinician(s) 216 can access these results in several ways—e.g., from the patient's user device 204, from the EHR in the one or more databases 212, or by logging in to the web application and reviewing the results for patients under their care.

The server 110 and the engagement model 304 thereon may be built and maintained in compliance with HIPAA safeguards. These include a separate, encrypted database layer accessed by the application over a private subnet for data and e-PHI (electronic Patient Health information) storage.

Additionally, communications between the server 210 and the platforms/user devices are secured over SSL. This includes the EHR, which may be communicated using REST APIs or other medical information exchange standards such as FHIR and ADT.

The engagement model 304 may be trained using analyzed data related to socio-demographics, behavioral health symptoms, and lifetime medical history for a plurality of patients. For diagnosed patients that did not initiate or otherwise obtain treatment, the model 304 may also be trained using the self-reported reasons for not doing so.

Self-reported reasons for not initiating treatment include that (i) the patient couldn't afford the cost of treatment; (ii) the patient thought they could manage without treatment; (iii) the patient didn't know where to go for treatment; (iv) the patient thought they might be committed or forced to take medication; (v) the patient didn't have time; (vi) the patient did not have enough health care coverage; (vii) the patient was concerned about their neighbors' opinion; (viii) the patient didn't think the treatment would help; (ix); the patient was concerned about confidentiality; (x) the patient didn't think they needed treatment; (xi) the patient was concerned about the treatment's effect on their job; (xii) the patient's health insurance didn't cover the treatment; (xiii) the patient didn't want others to find out about the treatment; (xiv) the patient didn't have transportation/location of treatment was too far; and (xv) for some other reason.

To train the engagement model 304, machine learning techniques may be applied to predict whether or not participants who self-identified as needing treatment obtained this treatment. Algorithms may be trained and tested using patient data from the U.S. National Survey on Drug Use and Health Series between 2008 and 2013, and validated on independent responses from 2014. From the tests, a number of patient-related risk factors for attrition may be identified, and a brief online questionnaire may be designed to identify participants that are at risk of not engaging in treatment. As mentioned above, the U.S. National Survey on Drug Use and Health Series is only one example of a dataset on which the engagement model 304 may be trained. The engagement model may be trained on a variety of other data sources in addition to or in lieu of the U.S. National Survey on Drug Use and Health Series.

In some embodiments, the engagement model 304 may rely on logistic regression methods. These methods are appropriate when covariates are correlated with one another, when predictors may only be sparsely endorsed, and when the outcome is binary (i.e., whether or not the patient will comply with a treatment regime). Additionally, the engagement model 304 may include an explicit penalty to avoid “overfitting” when the algorithm attempts to fit the noise instead of the underlying systematic relationship. In other embodiments, the engagement model 304 may rely on boosting techniques (e.g., extreme gradient boosting), discussed below.

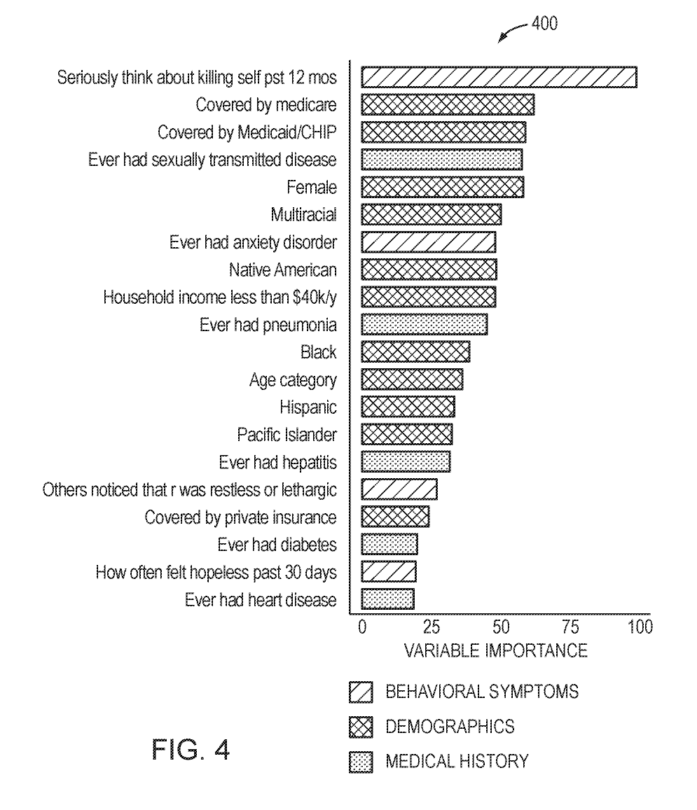

FIG. 4 presents a table 400 of the most important variables for predicting whether an individual who needs treatment will fail to initiate a treatment regime. The most informative variables for prediction have been determined to be whether a patient had previously thought about committing suicide, the patient's health insurance status (e.g., Medicare, or Medicaid/CHIP), whether the patient ever had a sexually transmitted disease, and the patient being female. Six of the top ten most influential variables in the engagement model 304 were sociodemographic, three were related to previous medical diagnoses, and one was a recent behavioral health symptom.

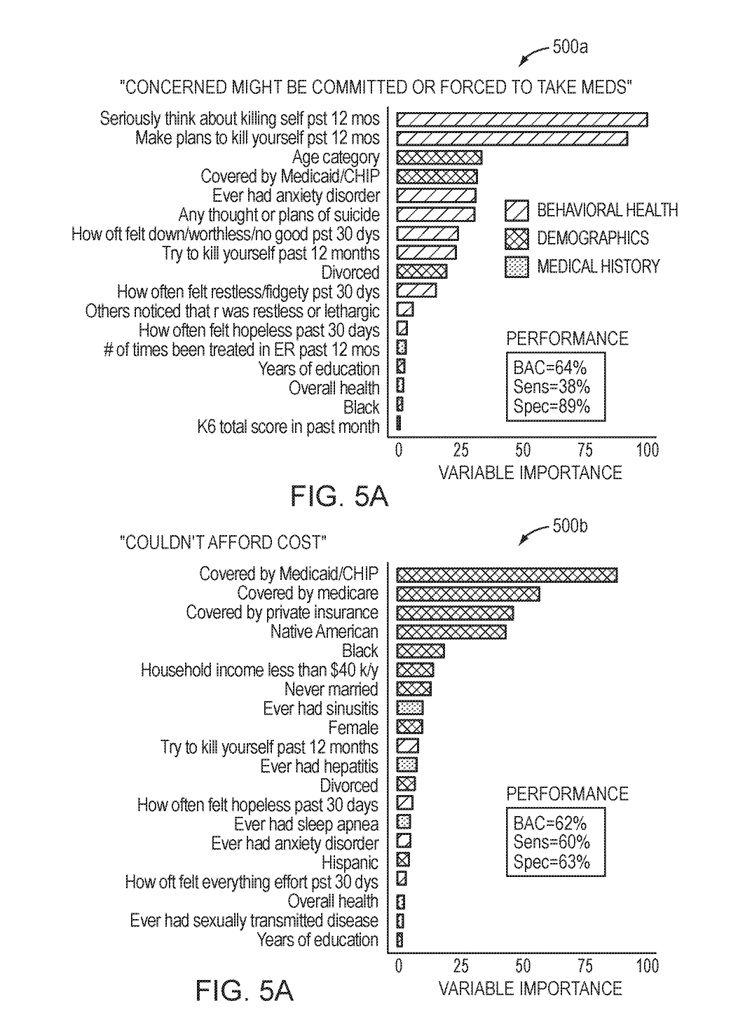

For each possible reason (i.e., each reason listed in table 400 of FIG. 4) the engagement model 304 may consider what factors are most important for causing a patient to endorse that particular reason. For example, for the reason of “thinking you might be committed or forced to take medications” the engagement model 304 may rely on suicide-related features. For “not being able to afford the cost” the engagement model relied on information about health insurance and racial ethnicity (Native American or African American). For “not having enough time/too busy for treatment” the engagement model relied on health insurance status (private or Medicare), female gender, and previous diagnoses of STD's or heart disease. This data is shown graphically in tables 500a-c of FIGS. 5a-c, respectively. Accordingly, the patient parameter(s) received from the patient 206 may include those listed in tables 500a-c of FIGS. 5a-c, respectively. These parameters listed are merely exemplary, and other factors, reasons, combinations thereof, and weighting schemas may be applied to accomplish the features of various embodiments described herein.

Based on the received patient parameters (and the subsequent prediction), clinicians may then tailor the patient's treatment regime(s) to make it at least more likely the patients will initiate their treatment regimes. Health care providers may determine a proportion of their population with whom they can afford to intervene (e.g., the decile at the highest risk of not initiating their treatment regimes), and then have care managers or social workers follow up with these patients to minimize concerns (e.g., cost of treatment, risks to patients, and where to go for care), and to improve initiation of the treatment regime.

Clinicians or other types of case managers may be guided by the reasons that the patient was predicted to endorse for not engaging in treatment, and use this knowledge to increase engagement through various care pathways. For example, patients who are too busy or live too far from a provider to obtain traditional face-to-face care may receive telemedicine referrals. Patients who are concerned about privacy or confidentiality may be directed to online CBT programs. Since low motivation, hypersomnia, and low energy are cardinal symptoms of depression, aggressive outreach attempts may be required to encourage some individuals to begin and remain in care.

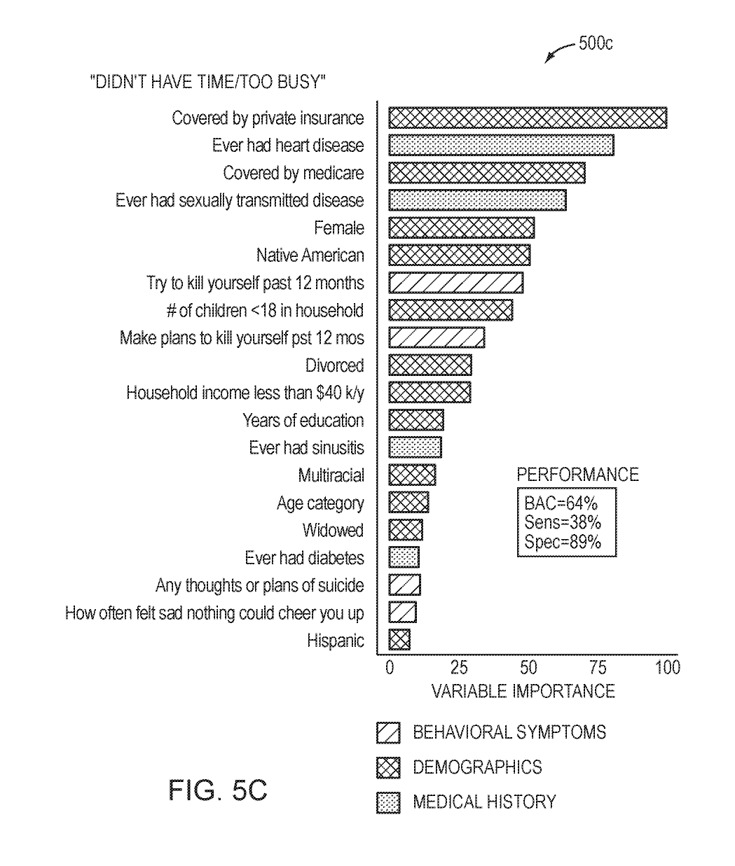

FIG. 6 illustrates a plot 600 relating to a patient that is predicted to not initiate a treatment regime. In this embodiment, plot 600 may be generated using a gradient boosting approach. Specifically, the plot 600 may be formed from a library and an ensemble for deriving individual-subject level variable importance. The output from the engagement model 304 in this embodiment may be the plot 600 which illustrates the impact of each feature for a particular individual, along with an overall prediction of whether the patient will initiate a treatment regime.

Specifically, the engagement model 304 may calculate the change in the log-odds prediction at each branch point in the route taken by the individual subject through an ensemble of trees, and attribute the change in prediction to the feature at the branch. The model 304 may sum the individual contributions for each feature to calculate a score representing the overall feature impact, which is unique to the observation. The “intercept” term simply refers to the log-odds prediction at the root of the first tree (i.e., before any branching has taken place).

A property of this boosting approach is that the sum of the intercept and feature impacts equals the overall log-odds prediction. These impacts are not static coefficients as in embodiments involving logistic regression. Rather, a feature's impact is dependent on the specific path through the ensemble of trees taken by an observation.

Referring back to FIG. 6, the x-axis of plot 600 may represent the probability of the response variable which, in plot 600, may refer to the probability that a patient will fail to initiate treatment. For example, values to the right of plot 600 correspond to high probabilities that a patient will fail to initiate a treatment regime.

At the top of plot 600 is the overall predicted probability of the engagement model 304 for a particular individual. The bars, from the bottom to the top of plot 600, illustrate a waterfall decomposition of each feature's contribution to the overall predicted probability. The bars may rank in order of the size of their contribution. The value inside the bar is the change in the log-odds attributable to that particular feature.

As seen in FIG. 6, the various features lead to a prediction that this particular patient is unlikely to initiate a treatment regime. This plot 600, along with other applicable data, may be communicated to a clinician device for viewing by a clinician.

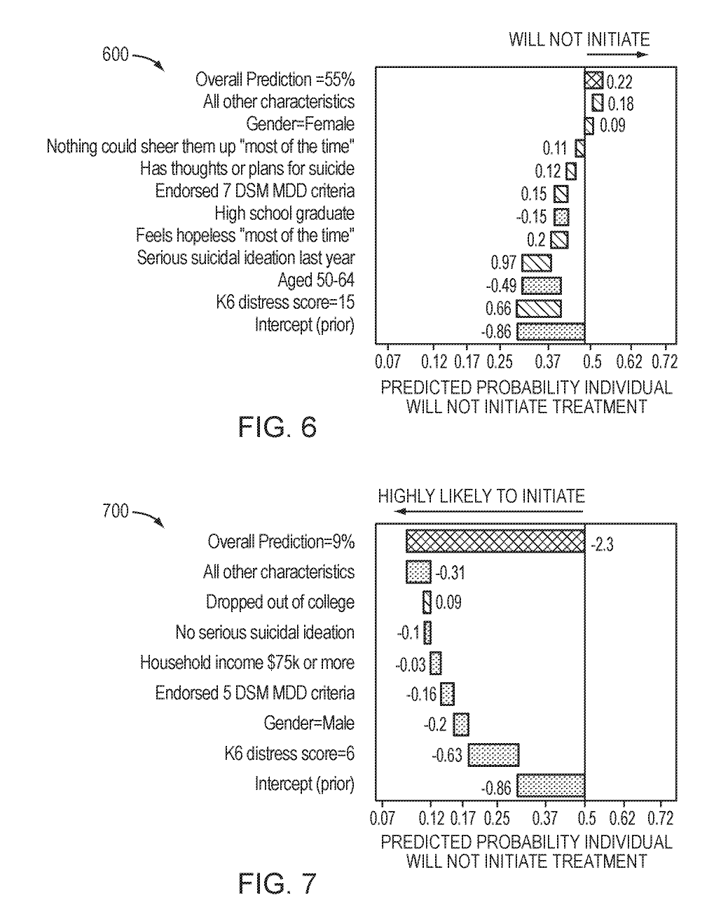

FIG. 7 illustrates a plot 700 relating to a patient that is predicted to initiate a treatment regime. Plot 700 may be generated similarly to plot 600 of FIG. 6. However, the patient parameters corresponding to the patient related to plot 700 suggests that the patient is highly likely to initiate treatment. This plot 700, along with other applicable data, may be communicated to a clinician device for viewing by a clinician.

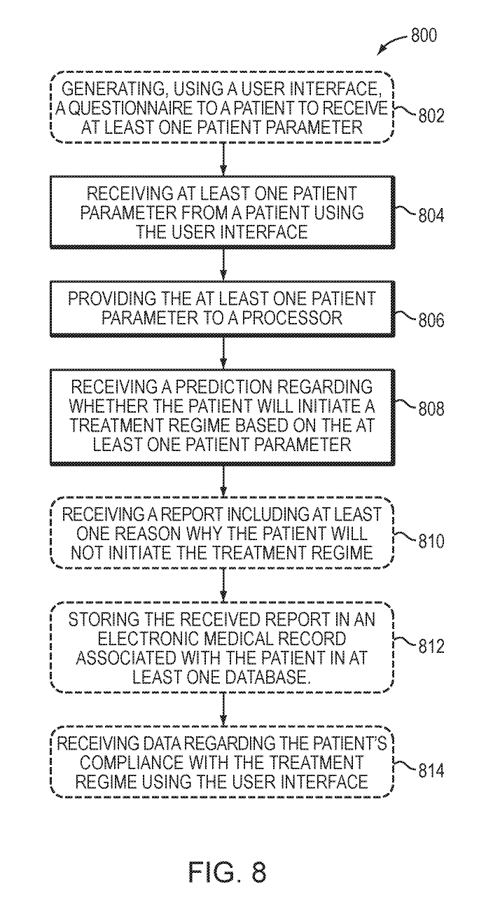

FIG. 8 depicts a flowchart of a method 800 for ensuring patient initiation of a treatment regime in accordance with one embodiment. Step 802 is optional and involves generating, using a user interface, a questionnaire to elicit from a patient at least one patient parameter. This step prompts the patient to provide certain types of information in a convenient, easy manner. The user interface may be similar to the user interface 202 of FIG. 2, for example.

Step 804 involves receiving at least one patient parameter from a patient using the user interface. The at least one patient parameter may include those discussed previously, such as data related to patient socio-demographics, patient behavioral health symptoms, and patient lifetime medical history. This data may be received by the questionnaire of step 802, if performed.

Step 806 involves providing the at least one patient parameter to a processor executing instructions stored on a memory and providing an engagement model. The engagement model may be similar to the engagement model 304 of FIG. 3, for example.

Step 808 involves receiving a prediction regarding whether the patient will initiate a treatment regime based on the at least one patient parameter. The engagement model may analyze the received patient parameter(s) in conjunction with training data to predict whether the patient is likely to initiate a treatment regime. The results of the engagement model may be communicated to a clinician via any suitable interface. The prediction may be presented as a binary output (e.g., “yes” or “no” regarding whether the patient will initiate the treatment regime), or may be presented as a probability, for example.

Step 810 is optional and involves receiving a report including at least one reason why the patient will not initiate the treatment regime. In some embodiments, the engagement model may generate a report regarding the prediction of whether or not the patient will initiate the treatment regime. The report maybe accompanied by one or more reasons the engagement model supplied a particular prediction.

Based on the reasons for non-initiation (and the weighting thereof), as well as other data available from questionnaires and/or EHR, the report may also include actionable recommendations for improving treatment regime initiation (and compliance). For example, if a barrier to initiation is cost, the report may recommend educating the patient on the benefits covered by their insurance. As another example, if a barrier is cost and time, the report may recommend that the provider explore tele-medicine alternatives because of scheduling flexibility and lack of travel accommodations. As yet another example, if a barrier is cost, and/or the patient is not covered by insurance per their EHR, the report may recommend considering cheaper digital therapy options.

The confidence of these recommendations can also be improved by machine learning and/or additional questionnaires. Accordingly, the confidence may be also depend on how much data is available for a particular patient. For example, a digital therapy recommendation discussed above may have a weaker confidence if the only information available is the potential to not initiate treatment based on cost. On the other hand, a digital therapy recommendation may have a higher confidence if data is available that indicates the patient has a mild condition, is young, is highly educated, or the like.

Step 812 is optional and involves storing the received report in an electronic medical record associated with the patient in at least one database. The generated report may be communicated to and stored in one or more databases along with the patient's EHR. Accordingly, clinicians can view outputs from the engagement model along with the patient's EHR to make more informed medical decisions. In some embodiments, the report may be stored on a server and/or database, and may also be transmitted to a patient-facing device. Accordingly, the output of the engagement model 304 may be communicated to any one or more of EHRs, handheld devices, servers, databases, or the like.

Step 814 is optional and involves receiving data regarding the patient's compliance with the treatment regime using the user interface. Accordingly, a patient may periodically provide data regarding their compliance with a treatment regime. A clinician may review this data to track their patient's progress with the treatment regime.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, or alternatively, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Having described several example configurations, various modifications, alternative constructions, and equivalents may be used without departing from the spirit of the disclosure. For example, the above elements may be components of a larger system, wherein other rules may take precedence over or otherwise modify the application of various implementations or techniques of the present disclosure. Also, a number of steps may be undertaken before, during, or after the above elements are considered.

Having been provided with the description and illustration of the present application, one skilled in the art may envision variations, modifications, and alternate embodiments falling within the general inventive concept discussed in this application that do not depart from the scope of the following claims.

What is claimed is:

1. A system for ensuring patient initiation of a treatment regime, the system comprising:
a user interface for receiving at least one patient parameter; and
a processor executing instructions stored on a memory and providing an engagement model, wherein the engagement model is configured to at least:
receive the at least one patient parameter,
predict whether a patient will initiate a treatment regime based on the at least one patient parameter, and
elevate the patient for further examination upon predicting the patient will not initiate the treatment regime.

2. The system of claim 1 wherein:
the user interface executes on a user device,
the processor executes on a server remote from the user device, and
the at least one patient parameter is cached on the user device until a connection is established between the user device and the server.

3. The system of claim 1 wherein the user interface is further configured to present a questionnaire to the patient to receive the at least one patient parameter from the patient.

4. The system of claim 1 wherein the engagement model is further configured to generate a report of the prediction, the generated report including at least one reason why the patient will not initiate the treatment regime.

5. The system of claim 4 wherein the generated report further includes at least one recommended treatment regime based on the at least one reason why the patient will not initiate the treatment regime.

6. The system of claim 1 wherein the at least one patient parameter includes at least one of medical history of the patient, socio-demographic data of the patient, and behavioral health symptoms of the patient.

7. The system of claim 1 wherein the user interface is further configured to receive data regarding the patient's compliance with the treatment regime.

8. The system of claim 1 wherein the prediction of whether the patient will initiate the treatment regime is further based on data from an electronic medical record associated with the patient.

9. The system of claim 1 wherein the engagement model predicts whether the patient will initiate a treatment regime by:
assigning a value to each of the plurality of patient parameters,
summing the assigned values to calculate a score, and
predicting whether the patient will initiate the treatment regime based on the score.

10. A method for ensuring patient initiation of a treatment regime, the method comprising:
receiving at least one patient parameter using a user interface;
providing the at least one patient parameter to a processor executing instructions stored on a memory and providing an engagement model; and
receiving a prediction regarding whether the patient will initiate a treatment regime based on the at least one patient parameter.

11. The method of claim 10 wherein
the user interface executes on a user device,
the processor executes on a server remote from the user device, and
the at least one patient parameter is cached on the user device until a connection is established between the user device and the server.

12. The method of claim 10 further comprising presenting, using the user interface, a questionnaire to the patient to receive the at least one patient parameter.

13. The method of claim 10 wherein the received prediction indicates the patient will not initiate the treatment regime, and the method further comprises receiving a report including at least one reason why the patient will not initiate the treatment regime.

14. The method of claim 13, wherein the received report further includes at least one recommended treatment regime based on the at least one reason why the patient will not initiate the treatment regime.

15. The method of claim 10 wherein the at least one patient parameter includes at least one of medical history of the patient, socio-demographic data of the patient, and behavioral health symptoms of the patient.

16. The method of claim 10 further comprising receiving data regarding the patient's compliance with the treatment regime using the user interface.

17. The method of claim 10 wherein the prediction of whether the patient will initiate the treatment regime is further based on data from an electronic medical record of the patient.

18. The method of claim 10 wherein the engagement model generates the prediction regarding whether the patient will initiate the treatment regime by:
assigning a value to each of the plurality of patient parameters,
summing the assigned values to calculate a score, and
predicting whether the patient will initiate the treatment regime based on the score.

19. A computer readable medium containing computer readable instructions for performing a method for ensuring patient initiation of a treatment, the medium comprising:
computer-readable instructions for receiving at least one patient parameter using a user interface;
computer-readable instructions for providing the at least one patient parameter to a processor executing instructions stored on a memory and providing an engagement model; and
computer-readable instructions for receiving a prediction regarding whether the patient will initiate a treatment regime based on the at least one patient parameter.

20. The computer readable medium of claim 17 wherein the at least one patient parameter includes at least one of medical history of the patient, socio-demographic data of the patient, and behavioral health symptoms of the patient.
